(19) United States
(12) Patent Application Publication
(10) Pub. No.: US 2003/0186938 A1
(43) Pub. Date: Oct. 2, 2003

(54) Water dispersible formulation of paroxetine

(76) Inventors: Ahmad Khalef Al-Deeb Al-Ghazawi, Waltham Cross (GB); David Philip Elder, Hertford (GB); Padma Meneaud, Knebworth (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/203,321

(22) PCT Filed: Feb. 9, 2001
(86) PCT No.: PCT/GB01/00569

(51) Int. Cl. A61K 31/724; A61K 31/452; A61K 9/20

(52) U.S. Cl. 514/58; 514/321; 424/465

(57) ABSTRACT

A water-dispersible formulation of paroxetine for immediate oral administration comprises a dry blend of paroxetine, a water-soluble dispersing agent, and a taste-masking agent, as a dispersible powder or moulded into a tablet.

The present invention relates to a novel composition containing a pharmaceutically active compound, and to the use of the composition in therapy. In particular, this invention is concerned with a formulation of paroxetine that is dispersible in water.

Pharmaceutical products with antidepressant and anti-Parkinson properties are described in U.S. Pat. No. 3,912,743 and U.S. Pat. No. 4,007,196. An especially important compound among those disclosed is paroxetine, the (−)trans isomer of 4-(4′-fluorophenyl)-3-(3′,4′-methylenedioxy-phenoxymethyl)-piperidine. This compound is used in therapy as the hydrochloride salt for the treatment and prophylaxis of inter alia depression, obsessive compulsive disorder (OCD) and panic.

Paroxetine hydrochloride has been described in the literature as a crystalline hemihydrate (see EP-A-0223403 of Beecham Group) and as various crystalline anhydrate forms (see WO96/24595 of SmithKline Beecham plc). These known forms are not ideally suited for all pharmaceutical applications because the known solid forms of paroxetine hydrochloride are relatively insoluble and are slow to dissolve completely.

However, for some patients swallowing a tablet can be difficult, whereas swallowing liquified medication is more easily carried out.

The present invention aims to satisfy the need for a liquid formulation of paroxetine hydrochloride. This achieved by providing a solid paroxetine formulation which is dispersible in water or an aqueuos medium for immediate administration, thus avoiding the need to store solutions or dispersions with risk of hydrolysis.

According to one aspect of the invention there is provided a dry blend of paroxetine, a water-soluble dispersing agent and a taste-masking agent.

The reference to paroxetine includes all forms of the compound in which paroxetine is available as a therapeutically effective agent. This includes paroxetine free base and pharmaceutically acceptable salts of paroxetine, especially paroxetine hydrochloride, particularly as the hemihydrate or one of the anhydrate forms.

The composition may be in powder form, especially with one or more conventional excipients, such as diluents, flavouring agents and sweeteners. Preferably a powder form is supplied as sealed sachets of the powder containing a unit dose of paroxetine. Alternatively the powder may be loaded into capsule shells, which are broken to add the powder to an aqueous carrier.

The composition may also be provided as a shaped composition such as a tablet, in which case the composition typically includes one or more conventional excipients for tablet formation, such as mould lubricants and disintegrants. Tablets may be formulated to disintegrate in water, for dispersion as a suspension for swallowing by drinking, or as bite-dispersion tablets which are broken in the mouth by biting and dispersed in saliva for swallowing.

Suitable dispersing agents include polyvinyl pyrrolidone (such as Crospovidone XL, from ISP International Corp), calcium carbonate (such as Cal-Carb, from Whittaker, Clark & Daniels), and sodium starch glycolate (such as Explotab, from Edward Mendell Co Inc). These are incorporated into the formulation, singularly or in combination, to disperse the active ingredient in water after break-up of a tablet or addition of a powder to water, and to maintain the active ingredient in a dispersed form.

Because of the bitter taste of paroxetine, when paroxetine is administered in a dosage form which is not swallowed whole, it is in practical terms essential that the composition also incorporates a taste masking agent to assist in patient compliance. Suitable taste masking agents includes potassium form polyacrylic acid ion exchange resins (such as Polacrilin K, from Rohm & Haas), β-cyclodextrin (such as Kleptose, from Roquette Inc), lecithin (such as Epikuron, from Lucas Meyer) and methacrylic acid copolymers (such as Eudragit L30D55, from Rohm & Haas).

The taste masking agents typically act by the formation of either an ion-exchange resin, inclusion complex, encapsulation or coating of the drug, to assist the patient to comply fully with the medication regime by swallowing the whole of the liquid dispersion.

Alternatively the taste masking agent may be an intense sweetener, such as those derived from fruit flavanoids.

The relative quantities of the dispersing agents may be adjusted to satisfy the desired balance of dispersability and taste masking. Also, the amount of dispersing agents relative to the other tableting excipients may be adjusted to suit the desired requirements for the rate of break-up of the tablet in water.

Typical excipients to make up the balance of the tablet formulation and to provide the requisite moldability and integrity of the tablet structure are conventional additives such as magnesium stearate and microcrystalline cellulose. The tablet may also contain sweeteners and flavourings to adjust the desired taste characteristics.

For use as a powder, the paroxetine, dispersing and taste masking agents may be blended as powders with other excipients such as solid diluents, flow control agents and desiccants, and then loaded into sachets or capsule shells by conventional means.

In an alternative procedure, the paroxetine is dispersed in a solution of a capsulateing material and spray dried before blending with other excipients for tabletting or filling powder containers.

The paroxetine hydrochloride used in this invention is preferably in the form of the crystalline hemihydrate (see EP-A-0223403). However other crystalline forms may also be used such as crystalline anhydrates (see WO96/24595), and other salts such as the maleate and acetate (see U.S. Pat. No. 3,912,743 and U.S. Pat. No. 4,007,196).

Therapeutic uses of the paroxetine composition of this invention include treatment of alcoholism, anxiety, depression, obsessive compulsive disorder, panic disorder, chronic pain, obesity, senile dementia, migraine, bulimia, anorexia, social phobia, pre-menstrual syndrome (PMS), adolescent depression, trichotillomania, dysthymia, and substance abuse, referred to below as “the Disorders”.

Accordingly, the present invention also provides:

the use of a composition of this invention for the treatment or prophylaxis of one or more of the Disorders; and

a method of treating one or more of the Disorders which comprises administering a composition of this invention to a person suffering from one or more of the Disorders.

The present invention is illustrated by the following Examples.

### EXAMPLE 1

| | gm |
|---|---|
| Paroxetine chloride hemihydrate | 22.80 |
| Polacrilin Potassium | 40.00 |
| Polyvinyl Pyrrolidone | 25.00 |
| Sweetener | 12.00 |
| Flavourings | 27.00 |
| Magnesium slearate | 2.50 |
| Microcrystalline Cellulose | 120.70 |
| 250.00 | |

The 250 g batch of the above materials were sieved, blended, and then subjected to compression in tablet moulds to form approx. 1000 tablets of approx. 250 mg.

Similarly, tablets were prepared from the formulations in Examples 1-7

### EXAMPLE 2

| | mg/tab |
|---|---|
| Paroxetine hydrochloride hemihydrate* | 22.76 |
| Polacrilin Potassium | 45.52 |
| Citric Acid | 5.00 |
| Polyvinylpyrrolidone | 25.00 |
| Calcium Carbonate | 25.00 |
| Flavour | 25.00 |
| Sweetener | 25.00 |
| Microcrystalline Cellulose | 74.22 |
| Magnesium Stearate | 2.50 |
| Total | 250.00 |

*equivalent to 20 mg paroxetine free base

### EXAMPLE 3

| | mg/tab |
|---|---|
| Paroxetine hydrochloride hemihydrate* | 22.76 |
| Polacrilin Potassium | 45.52 |
| Citric Acid | 5.00 |
| Polyvinylpyrroiidone | 25.00 |
| Calcium Carbonate | 15.00 |
| Flavour | 25.00 |
| Sweetener | 25.00 |
| Xylitol | 74.22 |
| Sodium Starch Glycolate | 10.00 |
| Magnesium Stearate | 2.50 |
| Total | 250.00 |

*equivalent to 20 mg paroxetine free base

### EXAMPLE 4

| | mg/tab |
|---|---|
| Paroxetine hydrochloride hemihydrate* | 22.76 |
| β-Cyclodextrin | 68.40 |
| Citric Acid | 5.00 |
| Polyvinylpyrrolidone | 25.00 |
| Calcium Carbonate | 25.00 |
| Flavour | 25.00 |
| Sweetener | 25.00 |
| Microcrystalline Cellulose | 51.34 |
| Magnesium Stearate | 2.50 |
| Total | 250.00 |

*equivalent to 20 mg paroxetine free base

### EXAMPLE 5

| | mg/tab |
|---|---|
| Paroxetine hydrochloride hemihydrate* | 22.76 |
| β-Cyclodextrin | 68.40 |
| Citric Acid | 5.00 |
| Polyvinylpyrrolidone | 25.00 |
| Calcium Carbonate | 15.00 |
| Flavour | 25.00 |
| Sweetener | 25.00 |
| Xylitol | 51.34 |
| Sodium Starch Glycolate | 10.00 |
| Magnesium Stearate | 2.50 |
| Total | 250.00 |

*equivalent to 20 mg paroxetine free base

### EXAMPLE 6

| | mg/tab |
|---|---|
| Paroxetine hydrochloride hemihydrate* | 22.76 |
| Lecithin | 45.52 |
| Citric Acid | 5.00 |
| Polyvinylpyrrolidone | 25.00 |
| Calcium Carbonate | 25.00 |
| Flavour | 25.00 |
| Sweetener | 25.00 |
| Microcrystalline Cellulose | 74.22 |
| Magnesium Stearate | 2.50 |
| Total | 250.00 |

*equivalent to 20 mg paroxetine free base

### EXAMPLE 7

| | mg/tab |
|---|---|
| Paroxetine hydrochloride hemihydrate* | 22.76 |
| Lecithin | 45.52 |
| Citric Acid | 5.00 |
| Polyvinylpyrrolidone | 25.00 |
| Calcium Carbonate | 15.00 |
| Flavour | 25.00 |
| Sweetener | 25.00 |
| Xylitol | 74.22 |
| Sodium Starch Glycolate | 10.00 |
| Magnesium Steatrate | 2.50 |
| Total | 250.00 |

*equivalent to 20 mg paroxetine free base

### EXAMPLE 8

| | gm |
|---|---|
| Paroxetine hydrochloride hemihydrate* | 22.76 |
| Methacrylic Acid Copolymer Type C | 1.14 |
| Talc | 0.35 |
| Triethyl Citrate | 0.14 |
| Citric Acid | 5.00 |
| Polyvinylpyrrolidone | 25.00 |
| Calcium Carbonate | 25.00 |
| Flavour | 25.00 |
| Sweetener | 25.00 |
| Microcrystalline Cellulose | 118.11 |
| Magnesium Stearate | 2.50 |
| Total | 250.00 |

*equivalent to 20 mg paroxetine free base

A suspension in water of paroxetine, methacrylic acid copolymer, talc and triethyl citrate from the above formulation was spray dried. The spray dried material and remaining excipients were sieved, blended, and then subjected to compression in tablet moulds to form approx. 1000 tablets of approx. 250 mg

### EXAMPLE 9

| | mg/tab |
|---|---|
| Paroxetine hydrochloride hemihydrate* | 22.76 |
| Methacrylic Acid Copolymer Type C | 1.14 |
| Talc | 0.35 |
| Triethyl Citrate | 0.14 |
| Citric Acid | 5.00 |
| Polyvinylpyrrolidone | 15.00 |
| Calcium Carbonate | 25.00 |
| Flavour | 25.00 |
| Sweetener | 25.00 |
| Xylitol | 118.11 |
| Sodium Starch Glycolate | 10.00 |
| Magnesium Stearate | 2.50 |
| Total | 250.00 |

*equivalent to 20 mg paroxetine free base

Using the procedure of Example 8, tablets were prepared from the above formulation.

1. A pharmaceutical composition which is a dry blend of:
paroxetine hydrochloride, another pharmaceutically acceptable salt of paroxetine, or paroxetine free base;
a water-soluble dispersing agent; and
a taste-masking agent.

2. A composition according to claim 1 which is in powder form.

3. A composition according to claim 1 which is a shaped composition including one or more conventional excipients for tablet formation.

4. A composition according to claim 1, 2 or 3 in which the dispersing agent is selected from polyvinyl pyrrolidone, calcium carbonate and sodium starch glycolate.

5. A composition according to claim 1, 2, 3 or 4 in which the taste masking agent is an intense sweetener.

6. A composition according to claim 5 in which the taste masking agent is selected from potassium form polyacrylic acid ion exchange resins, β-cyclodextrin, lecithin and methacrylic acid copolymers.

7. Use of a composition according to any one of claims 1 to 6 for the treatment or prophylaxis of one or more of the Disorders.

8. Method of treating one or more of the Disorders which comprises administering a composition according to any one of claims 1 to 6 to a person suffering from one or more of the Disorders
